(19) United States
(12) Patent Application Publication
(10) Pub. No.: US 2023/0274041 A1
(43) Pub. Date: Aug. 31, 2023

(54) METHOD OF MODELING USER-CUSTOMIZED ASSISTIVE TOOL, PROGRAM AND APPARATUS THEREFOR

(71) Applicant: SeeAnn Solution Co., Ltd., Incheon (KR)

(72) Inventors: Anna SEO, Incheon (KR); Youngjin JEONG, Daegu (KR)

(21) Appl. No.: 17/994,772

(22) Filed: Nov. 28, 2022

(30) Foreign Application Priority Data
Feb. 3, 2022 (KR) 10-2022-0014022

(51) Int. Cl. G06F 30/12; G06F 30/27

(52) U.S. Cl. CPC G06F 30/12; G06F 30/27; G06F 2111/16

(57) ABSTRACT

Proposed is an apparatus for modeling a user-customized assistive tool. The apparatus includes at least one processor; and a memory electrically connected to the processor to store at least one code executed by the processor. Accordingly, modeling of the assistive tool can be performed more easily, quickly and accurately.

### BACKGROUND

### 1. Technical Field

The present disclosure relates to a method, a program, and an apparatus for modeling a user-customized assistive tool to be worn on a disease or injury site.

### 2. Related Art

Human body tissues age over time. When diseases such as fractures, ligament injuries, degenerative arthritis, rheumatoid arthritis, tendinitis, trigger syndrome, and swan neck occur, medical or non-medical assistive tools are sometimes used to reduce the burden on the disease or injury site.

However, in order to properly apply the assistive tool, it is necessary to carefully analyze the body tissue of the wearer and perform a process for precisely wearing the assistive tool on the disease or injury site. In addition, it is necessary to reprocess the assistive tool according to the wear feedback of the assistive device wearer.

Because these processes require considerable time and manpower, there is a need to provide a method of providing a more convenient and sophisticated assistive tool to a user.

### Documents of Related Art

### Patent Document

(Patent Document 1) Korean Patent Publication No. 10-2015-0024982 (published on Mar. 10, 2015)

### SUMMARY

The present disclosure has been made in an effort to solve the problems in the related art, and one object of the present disclosure is to provide a method of modeling an assistive tool to be worn on a user’s body part based on an image.

Another object of the present disclosure is to provide a method of modeling a user-customized assistive tool according to the user’s body characteristics.

Still another object of the present disclosure is to provide a method of simulating wearing of a modeled assistive tool virtually.

The technical problems to be solved by the present disclosure are not limited to the aforementioned problems, and other technical problems not mentioned herein will be clearly understood from the following description by those skilled in the art to which the present disclosure pertains.

According to one aspect of the present disclosure, a method of modeling a user-customized assistive tool includes obtaining a three-dimensional body image of a user; providing one or more applicable shape templates of an assistive tool based on disease information of the user, skeletal information of a part where the assistive tool is worn, joint information, and characteristic information; and when the shape template of the assistive tool is selected, processing the selected shape template to be user-customized.

The providing of the shape templates may include providing a wear suitability of each of the shape templates based on a percentage.

The providing of the wear suitability may include providing the wear suitability based on a percentage by inputting the disease information, age information, gender information, skeletal information, joint information of the user, and the characteristic information of the part into a pre-learned fit analysis model.

The method may further include displaying the obtained body image on a three-dimensional basis.

In this case, the processing of the selected shape template may include selecting a plurality of reference points on a displayed body region on which an assistive tool is to be worn; and combining and displaying the selected shape template to the body region based on the plurality of selected reference points.

The processing of the selected shape template may include visually guiding a number and positions of the plurality of reference points based on a part on which the assistive tool is to be worn and a shape of the assistive tool.

The processing of the selected shape template may include adjusting a compression intensity level of the selected shape template, a size of a region covering the body region and an arrangement angle.

The method may further include outputting the processed shape template by using a three-dimensional printer after the adjusting.

According to another aspect of the present disclosure, there may be provided a program for modeling a user-customized assistive tool stored in the medium to execute the above-described method in combination with a computer that is hardware.

According to still another aspect of the present disclosure, an apparatus for modeling a user-customized assistive tool includes at least one processor; and a memory electrically connected to the processor to store at least one code executed by the processor.

The memory may store codes that, when executed, cause the processor to: obtain a three-dimensional body image of a user, provide one or more applicable shape templates of an assistive tool based on disease information of the user, skeletal information of a part where the assistive tool is worn, joint information, and characteristic information, and when the shape template of the assistive tool is selected, process the selected shape template to be user-customized

The processor may be configured to, when a wear suitability of each of the shape templates based on a percentage is provided, provide the wear suitability based on a percentage by inputting the disease information, age information, gender information, skeletal information, joint information of the user, and the characteristic information of the part into a pre-learned fit analysis model.

Other specific details of the present disclosure are included in the detailed description and drawings.

By providing a user-customized modeling method according to an embodiment of the present disclosure, an assistive tool to be worn on a user’s disease or injury site can be modeled simply and precisely, so that user convenience and device efficiency can be improved.

The effects of the present disclosure are not limited to the aforementioned effects, and other effects, which are not mentioned above, may be clearly understood by those skilled in the art to which the present disclosure pertains from the following descriptions.

### BRIEF DESCRIPTION OF THE DRAWINGS

FIG. 1 is a figure schematically illustrating an apparatus for modeling a user-customized assistive tool according to an embodiment of the present disclosure.

FIG. 2 is a block diagram illustrating the configuration of an apparatus for modeling a user-customized assistive tool according to an embodiment of the present disclosure.

FIG. 3 is a view illustrating a process of providing a shape template of an assistive tool applicable to a disease or injury site according to an embodiment of the present disclosure.

FIG. 4 is a view illustrating a process of selecting a reference point for positioning an assistive tool according to an embodiment of the present disclosure.

FIG. 5 is a view illustrating a process of adjusting the size and arrangement angle of a region covering a body region according to an embodiment of the present disclosure.

FIG. 6 is a flowchart illustrating a method of modeling a user-customized assistive tool according to an embodiment of the present disclosure.

FIG. 7 is a block diagram illustrating an apparatus for semi-automatically modeling a user-customized finger assistive tool according to an embodiment of the present disclosure.

FIG. 8 is a flowchart illustrating a method of semi-automatically modeling a user-customized finger assistive tool according to an embodiment of the present disclosure.

FIG. 9 is a block diagram illustrating an apparatus for semi-automatically modeling a user-customized upper extremity assistive tool according to an embodiment of the present disclosure.

FIG. 10 is a flowchart illustrating a method of semi-automatically modeling a user-customized upper extremity assistive tool according to an embodiment of the present disclosure.

FIG. 11 is a figure illustrating a portion of triangular polygons in 3D modeling.

### DETAILED DESCRIPTION

Like reference numerals refer to like elements throughout the specification. This specification does not describe all the elements of the embodiments, and the general contents of the related art or duplicative contents in the embodiments will be omitted. The terms ‘unit’, ‘module’, ‘member’, and ‘block’ used in the specification may be implemented by hardware or software. It is also possible that a plurality of units, modules, members, and blocks are implemented as one component, or one unit, module, member, or block includes a plurality of elements in accordance with the embodiments.

Throughout the specification, it will be understood that when an element is referred to as being “connected” to another element, it may be directly connected or indirectly connected to another element. The indirect connection includes a connection through a wireless communication network.

In addition, when some part “includes” some elements, unless explicitly described to the contrary, it means that other elements may be further included but not excluded.

Throughout the specification, when a member is located “on” another member, this includes not only a case in which a member is in contact with another member but also a case in which another member is present between the two members.

Terms, such as “first”, “second” etc, are for discriminating one component from another component, but the scope is not limited to the terms.

Singular forms are intended to include plural forms unless the context clearly indicates otherwise.

The reference characters used in the steps are used for the convenience of illustrating, and they do not mean the order of the steps and the steps may be generated in different orders, unless the order is specifically stated.

Hereinafter, the working principle and embodiments of the present disclosure will be described with reference to the accompanying drawings.

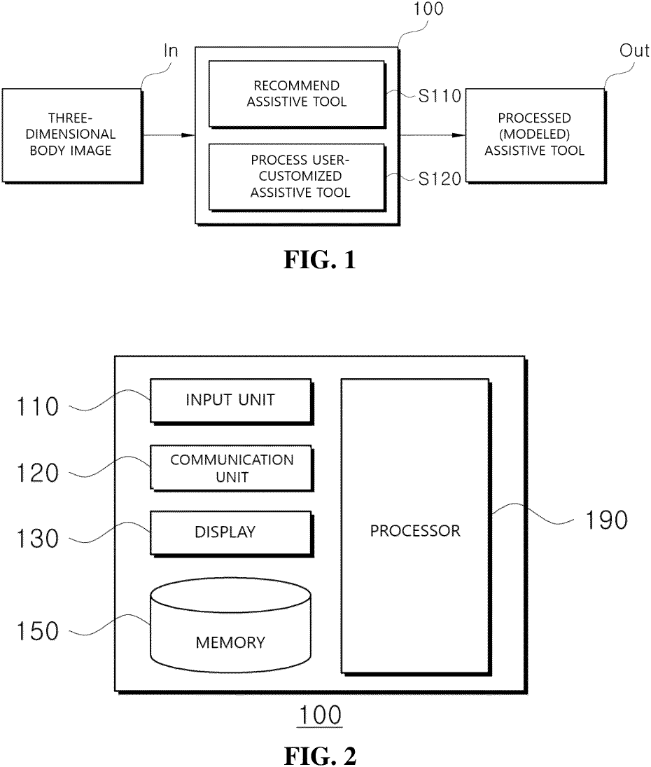

FIG. 1 is a figure schematically illustrating an apparatus 100 for modeling a user-customized assistive tool (hereinafter, referred to as a “modeling apparatus”) according to an embodiment of the present disclosure.

Referring to FIG. 1, the modeling apparatus 100 may provide an environment for visually confirming an assistive tool even though an assistive tool wearing subject does not directly visit a medical institution, and may provide a user with an environment in which the user can virtually experience wearing of the assistive tool.

The modeling apparatus 100 may be implemented as a computer or a portable terminal that can be accessed through a network. In this case, the computer may include, for example, a laptop computer, a desktop computer, a tablet PC, a slate PC, and the like equipped with a web browser. The portable terminal, which is a wireless communication device with guaranteed portability and mobility, may include, for example, all kinds of handheld-based wireless communication devices such as a personal communication system (PCS) terminal, a global system for mobile communications (GSM) terminal, a personal digital cellular (PDC) terminal, a personal handy-phone system (PHS) terminal, a personal digital assistant (PDA) terminal, an international mobile telecommunication (IMT)-2000 terminal, a code division multiple access (CDMA)-2000 terminal, a w-code division multiple access (W-CDMA) terminal, a wireless broadband Internet (WiBro) terminal, a smart phone and the like, and a wearable device such as a watch, a ring, a bracelet, an anklet, a necklace, glasses, a contact lens, a head-mounted-device (HMD), and the like.

The modeling apparatus 100 may receive the three-dimensional body image In of a user, and may output an assistive tool Out processed (modeled) through internal processes S110 and S120.

Specifically, the modeling apparatus 100 may display the three-dimensional body image In of the user, recommend an assistive tool according to the characteristics of a part where the assistive device is to be worn in process S110, and process a user-customized assistive tool in process S120. The modeling apparatus 100 may output the processed assistive tool.

FIG. 2 is a block diagram illustrating the configuration of the modeling apparatus 100 according to an embodiment of the present disclosure.

The modeling apparatus 100 may be implemented as an apparatus, a device, a PC, a tablet PC, a server, a cloud system, or the like. The modeling apparatus 100 may include an input unit 110, a communication unit 120, a display 130, a memory 150, and a processor 190. However, because the above components are not essential, the modeling apparatus 100 of the present specification may include more or fewer components.

The input unit 110, which is a module for obtaining a three-dimensional body image of a user, may include various input units, and obtain various 3D data formats (e.g., STL, OBJ, STEP, KGES, VRML, and the like) by using various functions (e.g., reconEasy).

The input unit 110 may include a hardware device for user input such as various buttons, a switch, a pedal, a keyboard, a mouse, a track-ball, various levers, a handle, a stick, and the like.

In addition, the input unit may include a graphical user interface (GUI) for user input such as a touch pad, that is, a software device. The touch pad may be implemented as a touch screen panel (TSP) and may form a layered structure with the display unit. When configured as a touch screen panel (TSP) forming a layered structure with a touch pad, the display 130 may also be used as the input unit 110.

The communication unit 120 may include at least one component that enables communication with an external device, and may include, for example, at least one of a short-range communication module, a wired communication module and a wireless communication module.

The short-range communication module may various short-range communication modules, such as a Bluetooth module, an infrared communication module, a radio frequency identification (RFID) communication module, a wireless local access network (WLAN) communication module, an NFC communication module, a Zigbee communication module, and the like, which transmit and receive signals in a short distance by using a wireless communication network.

The wired communication module may include various cable communication modules such as a universal serial bus (USB) module, a high definition multimedia interface (HDMI) module, a digital visual interface (DVI) module, a recommended standard-232 (RS-232) module, a power line communication module, a plain old telephone service (POTS), and the like as well as various wired communication modules such as a local area network (LAN) module, a wide area communication (WAN) module, a value added network (VAN) module, and the like.

The wireless communication module may include a wireless communication module supporting various wireless communication schemes such as global system for mobile communication (GSM), code division multiple access (CDMA), wideband code division multiple access (WCDMA), universal mobile telecommunications system (UMTS), time division multiple access (TDMA), long term evolution (LTE), and the like in addition to a Wi-Fi module and a wireless broadband (WiBro) module.

The display 130, which is a module for outputting various kinds of information, may output an operation process of the processor 190, a deep learning operation process, and the like to a screen, and may output various processing results. The display 130 may be implemented with a cathode ray tube (CRT), a digital light processing (DLP) panel, a plasma display panel, a liquid crystal display (LCD) panel, an electro luminescence (EL) panel, an electrophoretic display (EPD) panel, an electrochromic display (ECD) panel, a light emitting diode (LED) panel, an organic light emitting diode (OLED) panel, or the like, but is not limited thereto.

The memory 150 may be electrically connected to the processor 190, may store at least one code executed by the processor 190, and may be a module collectively referring to various types of storage.

The memory 150 may be located not only inside the modeling apparatus 100 but also outside. The memory 150 may store information necessary for performing an operation using machine learning, an artificial neural network, and the like. The memory 150 may store various learning models. The learning models may be used to infer a result value with respect to new input data other than learning data, and the inferred value may be used as a basis for a decision to perform a certain operation.

The learning models may be trained based on label information, and in order to increase learning accuracy, various backpropagation, cross entropy algorithm, and the like may be applied such that the loss function may have a target value.

The memory 150 may be implemented as at least one storage medium of a non-volatile memory device such as a cache, a read only memory (ROM), a programmable ROM (PROM), an erasable programmable ROM (EPROM), an electrically erasable programmable ROM (EEPROM), and a flash memory, a volatile memory device such as a random access memory (RAM), a hard disk drive (HDD), and a CD-ROM, but is not limited thereto. The storage unit may be a memory implemented as a chip separate from the processor 190 described above with respect to a control unit, or may be implemented as a single chip with the processor 190.

The memory 150 may store a code that, when executed through the processor 190, causes the processor 190 to perform various processing therein.

The processor 190 may be implemented as one or more, and even if expressed as a singular number, may be regarded as including a plurality of processors. The processor 190 may be a module that controls the components of the modeling apparatus 100. The processor 190 may refer to a data processing device embedded in hardware having a physically structured circuit to perform a function expressed as a code or an instruction included in a program. As an example of the data processing device embedded in the hardware as described above, there may be a processing device such as a microprocessor, a central processing unit (CPU), a processor core, a multiprocessor, an application-specific integrated circuit (ASIC), a field programmable gate array (FPGA), and the like, but the embodiment is not limited thereto. The processor 190 may separately include a learning processor for performing artificial intelligence operations, or may have a learning processor itself.

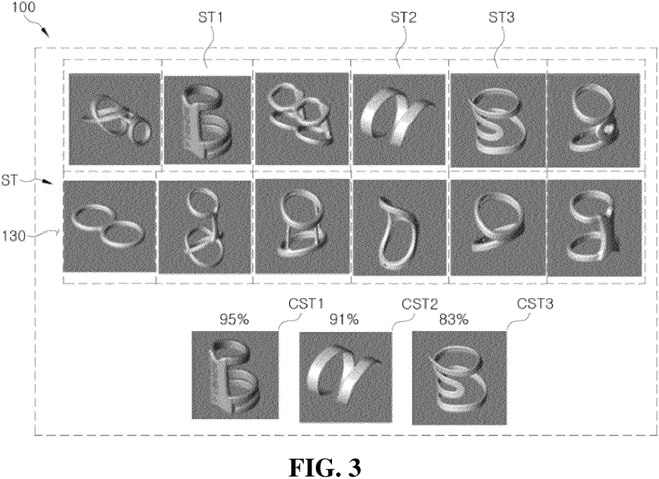

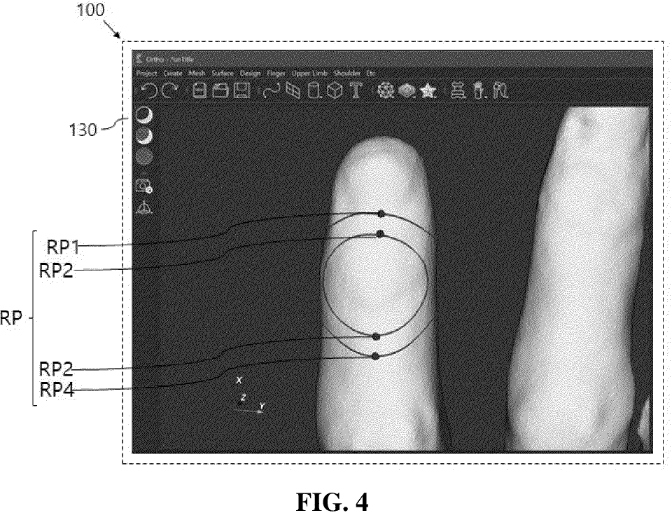

FIG. 3 is a view illustrating the modeling apparatus 100 that provides a shape template of an assistive tool applicable to a disease or injury site according to an embodiment of the present disclosure.

Referring to FIG. 3, the processor 190 may provide at least one shape template ST of an applicable assistive tool based on the user’s disease information, skeletal information of a part where the assistive tool is worn, joint information, and characteristic information. The shape template may vary depending on the body region to which the assistive tool is applied.

In this case, the user’s disease information may include information about a disease or injury site (e.g., a finger), name information of the disease or injury (e.g., fracture, abrasion, and the like), disease-related information (e.g., information about precautions, information about diet therapy, and the like), where skeletal information may include bone shape information, number information, strength information, and the like, joint information may include joint shape information, number information, location information, and the like, and characteristic information may include information on whether the assistive tool should not directly touch the disease or injury site, information on whether the compression strength should be less than or equal to a predetermined level, and the like. However, the embodiment is not limited thereto.

The processor 190 may recommend (e.g., CST1, CST2 and CST3) a plurality of shape templates ST1, ST2 and ST3 in consideration of wearing suitability after listing assistive tools applicable to disease or injury sites.

In this case, the processor 190 may provide (e.g., 95%, 91% and 83%) the wearing suitability of each shape template based on a percentage. In addition, the processor 190 may collect information on the most effective assistive tool for a corresponding disease and use the information to determine a numerical value of wearing suitability.

As an optional or additional embodiment, the processor 190 may provide the wearing suitability of each shape template based on a percentage based on the fit analysis model stored in the memory 150. The fit analysis model may be trained to receive the disease information, age information, gender information, skeletal information and joint information of the user, fit information when applying various user past cases, and characteristic information of the site, and output the wearing suitability based on a percentage. The trained model may be applied.

As an optional or additional embodiment, the processor 190 may output the shape template of the assistive tool wearing suitability to the display 130 in the order of wearing suitability, or may output the shape template of the assistive tool to the display 130 based on the user’s selection.

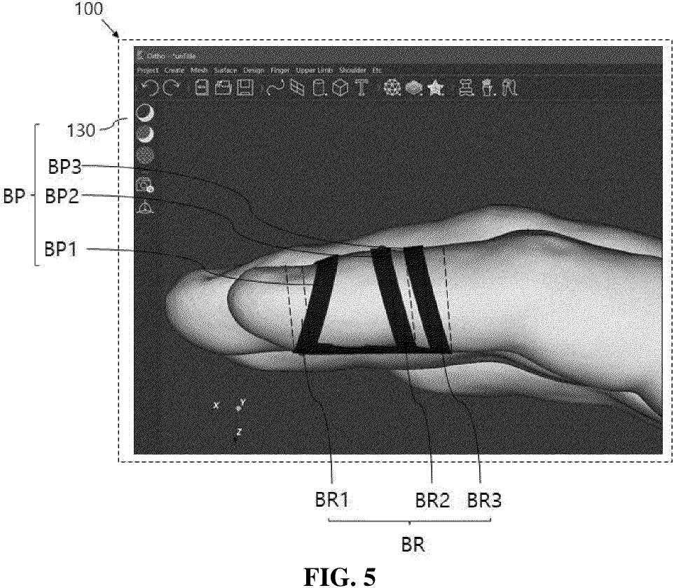

FIGS. 4 and 5 are views illustrating a process for virtually applying an assistive tool to a disease or injury site after determining a shape template of the assistive tool to be worn on the disease or injury site according to an embodiment of the present disclosure.

Specifically, FIG. 4 is a view illustrating a process of selecting a reference point for positioning an assistive tool according to an embodiment of the present disclosure. FIG. 5 is a view illustrating a process of adjusting the size and arrangement angle of a region covering a body region according to an embodiment of the present disclosure.

Referring to FIG. 4, the processor 190 may display the obtained body image on the display 130 based on three dimensions, and then select a plurality of reference points on the displayed body region on which the assistive tool is to be worn.

In this case, the plurality of reference points may be mapped to the assistive tool in advance according to the characteristics of the assistive tool, and may be selected by a user’s selection, but the embodiment is not limited thereto.

The processor 190 may visually guide the number and position of reference points based on a site where the assistive tool is to be worn and the shape of the assistive tool. For example, the processor 190 may visually guide the user that four reference points RP (RP1, RP2 and RP4) should be selected for the shape template CST1 of the selected assistive tool.

The processor 190 may adjust the position of the reference point by the user’s selection, and may guide the maximum moveable range of the reference point. As an optional embodiment, when exceeding the maximum moveable range of the reference point, the processor 190 may provide this as a notification and guide the user’s selection by expressing the maximum moveable range on the display 130.

The processor 190 may guide the selection sequence of the reference point. In the case of a finger injury, after guiding to first select the second reference point RP2, which is the central point, the processor 190 may guide to select the first reference point RP1 and the third reference point RP4. In addition, in this case, the first reference point RP1 may be the back surface of the finger, and the third reference point RP3 may also be the back surface of the finger, but the embodiment is not limited thereto.

The processor 190 may combine the selected shape template with the body region based on the plurality of selected reference points and output it on the display 130.

In an embodiment, the processor 190 may select a reference point from the model surface on a three-dimensional basis, and the triangle set T of the three-dimensional model may be expressed as following Equation 1.

$$τ \left(i\right) = \left[\begin{matrix}{p}_{0} \\ {p}_{1} \\ {p}_{2}\end{matrix}\right] , o = \left[\begin{matrix}x \\ y \\ z\end{matrix}\right] \qquad \text{[Equation 1]}$$

In Equation 1, t represents a triangular polygon composed of three vertices in the 3D model, T represents a set of t, v represents a vertex in the 3D model and has coordinates of (x, y, z). x, y and z represent coordinates of the vertex on the x-axis, y-axis and z-axis.

Equations 2 to 5 may be described with reference to FIG. 11.

When there is a straight line 1 passing through a point P₀ and having direction vector n, following Equation 2 to Equation 5 may be applied to express the reference point P through which 1 passes T as a formula.

$$\begin{matrix}{e}_{0} = {v}_{1} - {v}_{0} , & {e}_{1} - {v}_{2} - {v}_{0} , & h = n \times {e}_{1} , & a = {e}_{0} ⋅ h\end{matrix} \qquad \text{[Equation 2]}$$

In Equation 2, v₀ represents vertex 0, v₁ represents vertex 1, v₂ represents vertex 2, e₀ represents the vector of v₀ → v₁, e₁ represents the vector of v₀ → v₂, n represents the vector direction of straight line 1 in FIG. 11, and h represents the cross product of e1 and n, and a represents the dot product of e₀ and h.

Where a constraint condition [if a > -ESP and a < ESP, false. (ESP = 0.000001)] may be considered.

$$\begin{matrix}s = {P}_{0} - {v}_{0} , & u = \frac{1}{a} \times s ⋅ h\end{matrix} \qquad \text{[Equation 3]}$$

In Equation 3, P₀ represents an arbitrary vertex on straight line 1, s represents the vector of v₀ → P₀, u represents cross validation, and when 0 ≤ u ≤ 1, it means that straight line 1 intersects T. Wherein a constraint condition (if u < 0 or u > 1, false) may be considered.

$$q = s \times {e}_{o} \text{,} v = f \times n ⋅ g , t = f \times {e}_{1} ⋅ q \qquad \text{[Equation 4]}$$

In Equation 4, q represents the cross product of s and e₀, f is1/a, v represents cross-validation, and when v < 0 or u + v > 1, it means false. t represents ray cross-validation, and when t > ESP, it means true.

Where a constraint condition (if v < 0 or f+υ > 1, false) may be considered.

$$\begin{matrix}P = {P}_{0} + n \times t , & \left(t f t > E S P\right)\end{matrix} \qquad \text{[Equation 5]}$$

In Equation 5, P represents a point where straight line 1 passing through P₀ and having direction n intersects triangle t.

As described above, the processor 190 may select a reference point from the 3D model surface.

Referring to FIG. 5, the processor 190 may change a portion (ring shape) of the first recommended assistive tool from an initial position BR (BR1, BR2 and BR3) to an adjustment position BP (BP1, BP2 and BP3). The processor 190 may guide the arrangement angle change according to the skeletal information, joint information, and characteristic information of the disease or injury site, and the arrangement angle change may also be performed by a user input.

In this case, the processor 190 may determine the compression strength of the initial or adjusted shape template. For example, the processor 190 may calculate the compression suitability index A based on the material strength index P of the assistive tool, the diameter information BG of the disease or injury site, and the area information BK of the assistive tool surrounding the disease or injury site, and may determine the compression strength such that the calculated compression suitability index A meets a preset reference value range.

Accordingly, even when the actual user does not wear the assistive tool, the aftereffect or discomfort caused by the wearing of the assistive tool may be largely resolved.

In this case, the compression suitability index A may be calculated by the following Equation 6.

$$A = P \times \left(B K / B G\right) \qquad \text{[Equation 6]}$$

The processor 190 may output the processed shape template by using a 3D-based printer, and the 3D printer may be applied, but the embodiment is not limited thereto.

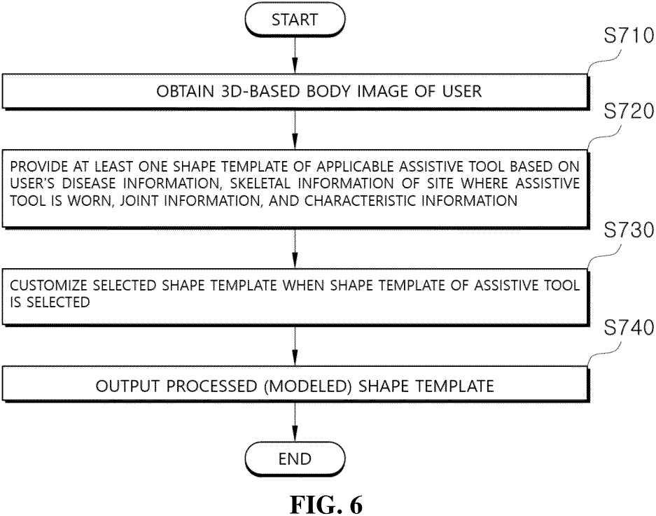

FIG. 6 is a flowchart illustrating a method of modeling a user-customized assistive tool according to an embodiment of the present disclosure.

First, in operation S710, the modeling apparatus 100 obtains the 3D-based body image of a user.

Thereafter, in operation S720, the modeling apparatus 100 may provide at least one shape template of the applicable assistive tool based on the user’s disease information, the skeletal information of the site where the assistive tool is worn, the joint information, and the characteristic information.

The modeling apparatus 100 may display the wearing suitability of each shape template based on a percentage.

Thereafter, when the shape template of the assistive tool is selected, in operation S730, the modeling apparatus 100 may customize the selected shape template.

The modeling apparatus 100 may guide the user’s selection by providing a user interface for selecting a plurality of reference points on a displayed body region on which the assistive tool is to be worn, and based on the plurality of selected reference points, may display the selected shape template in combination with the body region.

The modeling apparatus 100 may visually guide the number and positions of reference points based on the site where the assistive tool is to be worn and the shape of the assistive tool, and may adjust the compression strength of the selected shape template, the size of the region covering the body region, and the arrangement angle.

Thereafter, in operation S740, the modeling apparatus 100 may output a finally processed (modeled) shape template.

The method of modeling a user-customized assistive tool according to an embodiment of the present disclosure described above may be implemented as a program (or application) to be executed in combination with a computer which is hardware, and stored in a medium. In this case, the computer may be the modeling apparatus 100 described above.

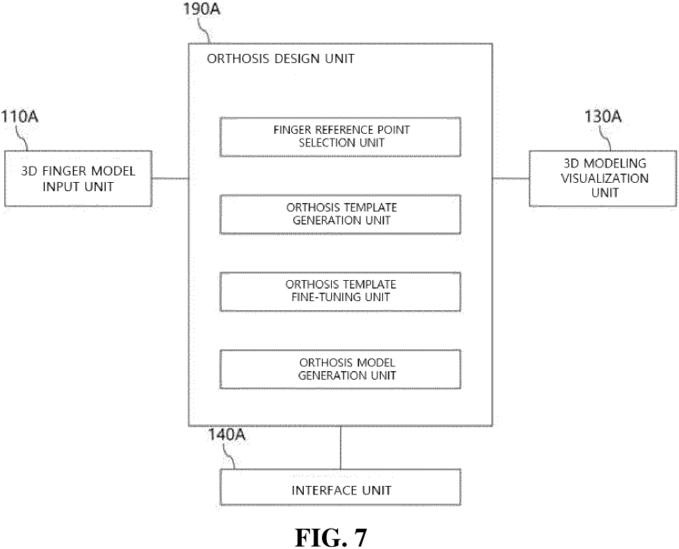

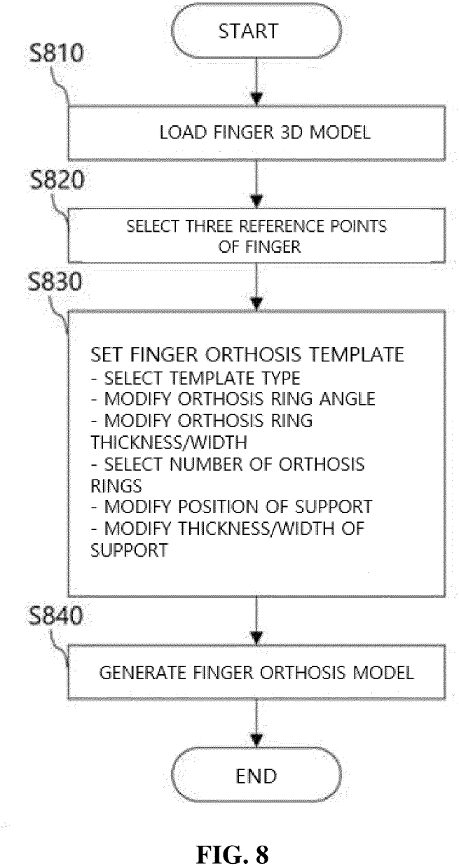

FIG. 7 is a block diagram illustrating an apparatus for semi-automatically modeling a user-customized finger assistive tool according to an embodiment of the present disclosure. FIG. 8 is a flowchart illustrating a method of semi-automatically modeling a user-customized finger assistive tool according to an embodiment of the present disclosure.

The modeling apparatus of FIG. 7 may be the above-described modeling apparatus 100, and a 3D finger model input unit 110A may be included in the above-described input unit 110. An orthosis design unit 190A may be included in the above-described processor 190, and a 3D modeling visualization unit 130A may be included in the display 130 described above. An interface unit 140A may include various interface modules.

When a 3D finger model is input, the orthosis design unit 190A may select a finger reference point, generate an orthosis template, fine-tune the orthosis template, and finally generate an orthosis model.

The orthosis design unit 190A may display the completed assistive tool model through the three-dimensional modeling visualization unit 130A.

Referring to FIG. 8, the modeling apparatus may load a finger 3D model in operation S810, and select three reference points of the finger in operation S820.

Thereafter, in operation S830, the modeling apparatus may set the finger orthosis template. In detail, the modeling apparatus may select the template type, modify the orthosis ring angle, modify the orthosis ring thickness/width, select the number of orthosis rings, modify the position of a support, and modify the thickness/width of the support.

In operation S840, the modeling apparatus may finally generate the finger orthosis model.

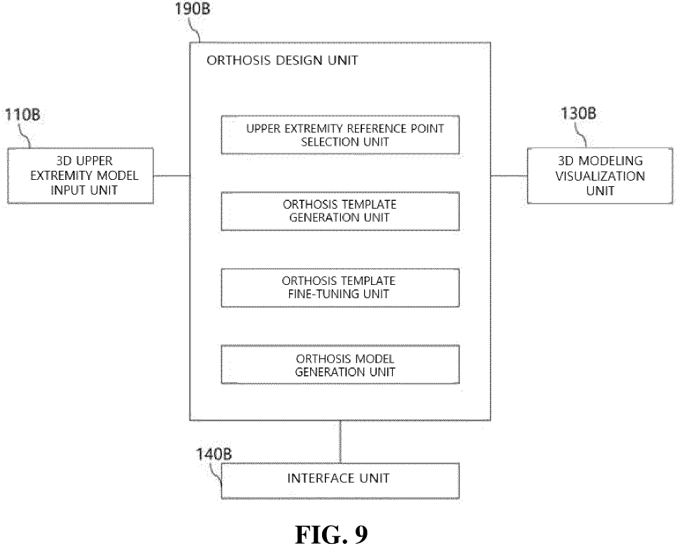

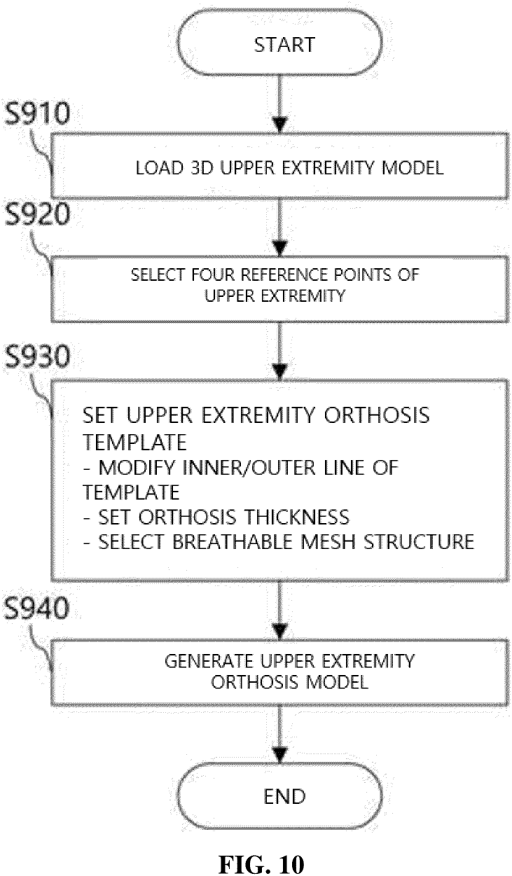

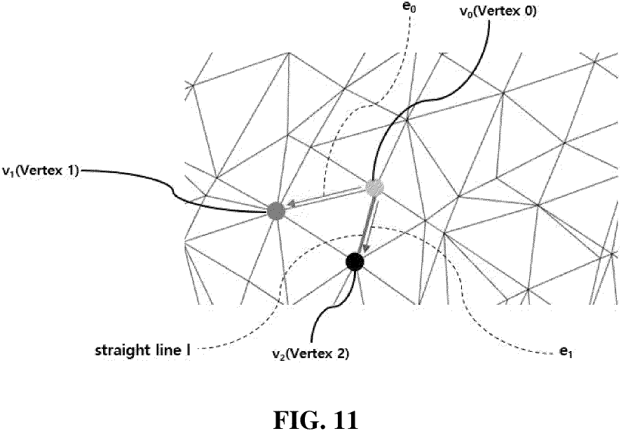

FIG. 9 is a block diagram illustrating an apparatus for semi-automatically modeling a user-customized upper extremity assistive tool according to an embodiment of the present disclosure. FIG. 10 is a flowchart illustrating a method of semi-automatically modeling a user-customized upper extremity assistive tool according to an embodiment of the present disclosure.

The modeling apparatus of FIG. 9 may be the modeling apparatus 100 described above, and a 3D upper extremity model input unit 110B may be included in the input unit 110 described above. An orthosis design unit 190B may be included in the above-described processor 190, and a 3D modeling visualization unit 130B may be included in the display 130 described above. An interface unit 140B may include various interface modules.

When a 3D upper extremity model is input, the orthosis design unit 190B may select an upper extremity reference point, generate an orthosis template, fine-tune the orthosis template, and finally generate an orthosis model.

The orthosis design unit 190B may display the completed assistive tool model through the three-dimensional modeling visualization unit 130B.

Referring to FIG. 10, the modeling apparatus may load an upper extremity 3D model in operation S910, and select four reference points of the upper extremity in operation S920.

Thereafter, the modeling apparatus may set the upper extremity orthosis template in operation S930. In detail, the modeling apparatus may modify the inner/outer outline of the template, set the orthosis thickness, and select a breathable mesh structure.

Finally, the modeling apparatus may generate an upper extremity orthosis model in operation S940.

As described above, in order for a computer to read a program and execute the methods implemented by the program, the program may include code coded in a computer language, such as C, C++, Python, JAVA, machine language, and the like, which can be read by a processor (CPU) of a computer through a device interface of the computer. The code may include a functional code related to a function or the like that defines the functions necessary to execute the methods and include an execution procedure related control code necessary for a processor of the computer to execute the functions according to a predetermined procedure. In addition, such code may further include memory reference related code as to whether additional information or media needed to cause the processor of the computer to execute the aforementioned functions should be referred to at a location (address) of the internal or external memory of the computer. In addition, when the processor of the computer needs to communicate with any other computer or server at a remote location in order to execute the functions, the code may further include communication-related codes for how to communicate with any other remote computer or server using the communication module of the computer, and what information or media to transmit/receive during communication.

The operations of a method or algorithm described in connection with the embodiments of the present disclosure may be embodied directly in hardware, in software modules executed in hardware, or in a combination of both. The software module may be reside on a random access memory (RAM), a read only memory (ROM), an erasable programmable ROM (EPROM), an electrically erasable programmable ROM (EEPROM), a flash memory, a hard disk, a removable disk, or any other form of computer readable recording medium known in the art to which the disclosure pertains.

While the present disclosure has been described with reference to exemplary embodiments, it will be apparent to those skilled in the art that various changes and modifications may be made without departing from the spirit and scope of the present disclosure. Therefore, it should be understood that the above embodiments are not limiting, but illustrative.

1. A method performed by an apparatus for modeling a user-customized assistive tool, the method comprising:
obtaining a three-dimensional body image of a user;
providing one or more applicable shape templates of an assistive tool based on disease information of the user, skeletal information of a part where the assistive tool is worn, joint information, and characteristic information; and
when the shape template of the assistive tool is selected, processing the selected shape template to be user-customized.

2. The method of claim 1, wherein the providing of the shape templates includes:
providing a wear suitability of each of the shape templates based on a percentage.

3. The method of claim 2, wherein the providing of the wear suitability includes:
providing the wear suitability based on a percentage by inputting the disease information, age information, gender information, the skeletal information, the joint information of the user, and the characteristic information of the part into a pre-learned fit analysis model.

4. The method of claim 3, further comprising:
displaying the obtained body image on a three-dimensional basis,
wherein the processing of the selected shape template includes:
selecting a plurality of reference points on a displayed body region on which an assistive tool is to be worn; and
combining and displaying the selected shape template to the body region based on the plurality of selected reference points.

5. The method of claim 4, wherein the processing of the selected shape template includes:
visually guiding a number and positions of the plurality of reference points based on a part on which the assistive tool is to be worn and a shape of the assistive tool.

6. The method of claim 5, wherein the processing of the selected shape template includes:
adjusting a compression intensity level of the selected shape template, a size of a region covering the body region and an arrangement angle.

7. The method of claim 6, further comprising:
outputting the processed shape template by using a three-dimensional printer after the adjusting.

8. A program for modeling a user-customized assistive tool stored in the medium to execute the method of claim 1 in combination with a computer that is hardware.

9. An apparatus for modeling a user-customized assistive tool, the apparatus comprising:
at least one processor; and
a memory electrically connected to the processor to store at least one code executed by the processor,
wherein the memory stores codes that, when executed, cause the processor to: obtain a three-dimensional body image of a user, provide one or more applicable shape templates of an assistive tool based on disease information of the user, skeletal information of a part where the assistive tool is worn, joint information, and characteristic information, and when the shape template of the assistive tool is selected, process the selected shape template to be user-customized.

10. The apparatus of claim 9, wherein the processor is configured to, when a wear suitability of each of the shape templates based on a percentage is provided, provide the wear suitability based on a percentage by inputting the disease information, age information, gender information, the skeletal information and the joint information of the user, and the characteristic information of the part into a pre-learned fit analysis model.
